(19) United States
(12) Patent Application Publication
(10) Pub. No.: US 2019/0184064 A1
(43) Pub. Date: Jun. 20, 2019

(54) Composition for Soft Tissue Augmentation Providing Protection from Infection

(71) Applicants: Robert DiLuccio, Haymarket, VA (US); Paul Lorenc, New York, NY (US); Randy Milby, Newark, DE (US)

(72) Inventors: Robert DiLuccio, Haymarket, VA (US); Paul Lorenc, New York, NY (US); Randy Milby, Newark, DE (US)

(21) Appl. No.: 15/765,090

(22) PCT Filed: Sep. 30, 2016
(86) PCT No.: PCT/US16/54926

§ 371 Date: Mar. 30, 2018

Related U.S. Application Data
(60) Provisional application No. 62/402,407, filed on Sep. 30, 2016.
(60) Provisional application No. 62/236,463, filed on Oct. 2, 2015.

(51) Int. Cl. A61L 27/54; A61L 27/38; A61L 27/20

(52) U.S. Cl. CPC A61L 27/54; A61L 27/3834; A61L 27/20; A61L 2300/252; A61L 2430/34; A61L 2300/216; A61L 2300/414; A61L 2400/06; A61L 2300/404

(57) ABSTRACT

A therapeutic filler composition and method of using the same for augmenting soft tissue is provided. The therapeutic filler composition may include an anti-microbial first component, a carrier, and a cross-linking agent, wherein the carrier is cross-linked with the cross-linking agent and the first component is combined with the cross-linked carrier into a combination having a therapeutic effect.

### RELATED APPLICATION DATA

This application claims priority from PCT Patent Application No. PCT/US16/54926 filed Sep. 30, 2016, and U.S. Provisional Applications Ser. No. 62/236,463 filed on Oct. 2, 2015 and Ser. No. 62/402,407 filed on Sep. 30, 2016.

### FIELD OF INVENTION

This application relates in general to a composition capable of being used in surgical procedures. Specifically, this application relates to an anti-microbial composition capable of being used for the augmentation of a patient's soft tissue.

### BACKGROUND OF THE INVENTION

Along with a trend toward living a longer, healthier life, patients are looking to physicians to help them achieve a more youthful appearance. The skin's natural aging process manifests contour changes and rhytids secondary to the depletion of subcutaneous fat and the loss of dermal collagen. Traditionally, rejuvenation has been achieved with a face-lift by surgically tightening the skin. Today, a multitude of minimally invasive procedures are aimed at rejuvenation without the risk, recovery time, and expense of major surgery. The development and popularity of BOTOX® has opened the door for equally noninvasive, adjunctive treatment of dynamic rhytids and soft tissue augmentation. Soft tissue augmentation has become a popular means of addressing issues such as, for example, contour defects that result from aging, photo-damage, trauma and/or scarification, or disease. A number of filling agents exist in the armamentarium.

Although most synthetic injectable facial fillers that have a permanent effect are generally non-toxic, they are known to turn immunogenic in tissues eliciting a host response. Inflammatory nodules likely caused by a low-grade infection may develop at the sites of injection affecting the longevity of the filler due to differences in their composition, and in chemical and biological characteristics. It is therefore desirable to have a non-toxic filler composition which can prolong the life of the filler by resisting infections.

### BRIEF SUMMARY

The present disclosure provides a non-toxic filler composition useful in soft tissue augmentation and methods of using the same.

In one embodiment the composition for augmenting the soft tissue is provided. The composition may include an anti-microbial agent, at least one growth factor, at least one stem cell, and a carrier. The anti-microbial agent in the composition may be Taurolidine and it may impart a prophylactic effect for the filler composition and prolong the useful life of the filler composition. The at least one growth factor in the composition may include transforming growth factor-beta (TGF-β) or vascular epithelial growth factor (VEGF) or epidermal growth factor (EGF), fibroblast growth factor (FGF) or bone morphogenetic proteins (BMPs) or platelet-derived growth factor (PDGF) or a combination of one or more of these growth factors. The stem cells may be autologous or non-autologous and the carrier may be a hyaluronic acid mix consisting of hyaluron and a sugar.

In another embodiment, a method of preparing the composition for soft tissue augmentation is provided. The method may include the steps of providing a first component comprising an anti-microbial agent, providing a second component comprising at least one growth factor, stem cells, and a carrier; wherein the carrier is cross-linked with a cross-linking agent and combining the first component and the second component so that the anti-microbial in the first component improves the useful life of the composition when injected in a patient's target tissue.

In another embodiment, a method of using the composition for soft tissue augmentation is provided. The method comprising preparing a therapeutic filler composition to a patient wherein the composition includes a combination of a first component comprising an anti-microbial agent and a second component comprising at least one growth factor, stem cells, and a carrier, wherein the carrier is cross-linked with a crosslinking agent and using the cross-linked carrier to deliver the composition to a target tissue and administering the composition to a subject in need of soft tissue augmentation.

These and other objects, features and advantages of the present invention will become apparent from the following detailed description of illustrative embodiments thereof.

### DESCRIPTION OF THE DRAWINGS

FIG. 1 is a graphic illustration of the thixotropic properties of the formulations with 3% drug as a function of frequency sweep.

### DETAILED DESCRIPTION

### Definitions

By way of example, as used in the specification or claims, “soft tissue” refers to any tissue supporting, surrounding or connecting structures of the body of a patient. In some embodiments the “soft tissue” specifically excludes the bone tissue. Also, by way of example, as used in the specification or claims, “soft tissue augmentation” refers to any procedure that amplifies the volume of soft tissue including, but not limited to, cosmetic, medical, or reconstructive procedures.

To the extent that the term “therapeutic effect” is used in the specification or claims, it is intended to refer to an effect sufficient to produce a measurable increase in soft tissue augmentation in the patient and/or a prolonged effect and increased collagen production in comparison to existing approaches (for example, an improvement of up to about 50%). The term “prophylactic effect” as used in the specification or claims refer to the preventive effect of the composition from risks of microbial infections.

The term “patient” as used in the specification or claims is intended, for example, to refer broadly to mammalian subjects, preferably humans receiving medical attention (for example, diagnosis, monitoring, etc.), care or treatment.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” To the extent that the term “substantially” is used in the specification or the claims, it is intended to take into consideration the degree of precision available or prudent in manufacturing. To the extent that the term “selectively” is used in the specification or the claims, it is intended to refer to a condition of a component wherein a user of the apparatus may activate or deactivate the feature or function of the component as is necessary or desired in use of the apparatus. As used in the specification and the claims, the singular forms “a,” “an,” and “the” include the plural. Finally, where the term “about” is used in conjunction with a number, it is intended to include ±10% of the number. In other words, “about 10” may mean from 9 to 11.

The composition as taught by the present disclosure includes a first component comprising an anti-microbial agent and a second component comprising at least one growth factor, stem cells and a carrier which is cross-linked with a cross-linking agent. The composition when administered to a subject in need thereof imparts one or more of the following advantages to the soft tissue augmentation procedure. The advantages include (i) improving or accelerating one or more processes of wound healing; (ii) prevent microbial infection at the site of injection; (iii) prolong the life of the filler at the target site; and (iv) improve the overall therapeutic effectiveness of the procedure.

Use of the Composition to Improve the Wound Healing Processes

Since the processes that can help promote longevity of a filler depends on the robustness of wound repair and collagen deposition, the composition of the present disclosure can improve or promote one or more of the following wound healing processes: (i) the inflammatory phase, (ii) the proliferative phase, and (iii) the maturational phase.

The first stage of wound healing, namely the inflammatory phase is characterized by hemostasis and inflammation. Collagen exposed during wound formation activates the clotting cascade (both the intrinsic and extrinsic pathways), initiating the inflammatory phase. After injury to tissue occurs, the cell membranes, damaged from the wound formation, release thromboxane A2 and prostaglandin 2-alpha that are potent vasoconstrictors. This initial response helps to limit hemorrhage. After a short period, capillary vasodilatation occurs secondary to local histamine release, and the inflammatory cells are able to migrate to the wound bed. The timeline for cell migration in a normal wound healing process is predictable.

Platelets, the first response cell, release multiple chemokines, including epidermal growth factor (EGF), fibronectin, fibrinogen, histamine, platelet-derived growth factor (PDGF), serotonin, and von Willebrand factor. These factors help stabilize the wound through clot formation. These mediators act to control bleeding and limit the extent of injury. Platelet degranulation also activates the complement cascade, specifically C5a, which is a potent chemo-attractant for neutrophils.

The inflammatory phase continues, and more immune response cells migrate to the wound. The second response cell to migrate to the wound, the neutrophil, is responsible for debris scavenging, complement-mediated opsonization of bacteria, and bacteria destruction via oxidative burst mechanisms (that is, superoxide and hydrogen peroxide formation). The neutrophils kill bacteria and decontaminate the wound from foreign debris.

The next cells present in the wound are leukocytes and macrophages (monocytes). The macrophage, also referred to as the orchestrator, is essential for wound healing. Numerous enzymes and cytokines are secreted by the macrophage. These include, for example, collagenases (which debride the wound), interleukins and tumor necrosis factor (TNF) (which stimulate fibroblasts (produce collagen) and promote angiogenesis), and transforming growth factor (TGF) (which stimulates keratinocytes). This step marks the transition into the process of tissue reconstruction, that is, the proliferative phase.

The second stage of wound healing is the proliferative phase. Epithelialization, angiogenesis, granulation tissue formation, and collagen deposition are the principal steps in this anabolic portion of wound healing. Epithelialization occurs early in wound repair. If the basement membrane remains intact, the epithelial cells migrate upwards in the normal pattern. This is equivalent to a first-degree skin burn. The epithelial progenitor cells remain intact below the wound, and the normal layers of epidermis are restored in 2-3 days. If the basement membrane has been destroyed, similar to a second- or third-degree burn, then the wound is re-epithelialized from the normal cells in the periphery and from the skin appendages, if intact (for example, hair follicles, sweat glands).

Angiogenesis, stimulated by TNF-alpha, is marked by endothelial cell migration and capillary formation. The new capillaries deliver nutrients to the wound and help maintain the granulation tissue bed. The migration of capillaries into the wound bed is critical for proper wound healing. The granulation phase and tissue deposition require nutrients supplied by the capillaries, and failure for this to occur results in a chronically unhealed wound. Mechanisms for modifying angiogenesis are under study and have significant potential to improve the healing process.

The final part of the proliferative phase is granulation tissue formation. Fibroblasts differentiate and produce ground substance and then collagen. The ground substance is deposited into the wound bed, and collagen is then deposited as the wound undergoes the final phase of repair. Many different cytokines are involved in the proliferative phase of wound repair. The steps and the exact mechanism of control have not been elucidated. Some of the cytokines include, for example, PDGF, insulin-like growth factor (IGF), and EGF.

The final phase of wound healing is the maturational phase. The wound undergoes contraction, ultimately resulting in a smaller amount of apparent scar tissue. The entire process is a dynamic continuum with an overlap of each phase and continued remodeling. The wound reaches maximal strength at one year, with a tensile strength that is 30% of normal skin. Collagen deposition continues for a prolonged period, but the net increase in collagen deposition plateaus after 21 days.

In existing approaches that promote wound healing, cytokines have a limited role in clinical practice. For example, the only currently available commercial product proven to be efficacious in randomized, double blind-studies is Platelet-derived growth factor (PDGF), available as recombinant human PDGF-BB. In multiple studies, recombinant human PDGF-BB has been demonstrated to reduce healing time and improve the incidence of complete wound healing in stage III and IV ulcers. Besides PDGF another family of exogenous growth factor that is known to have broader and significant effects in the promoting of wound healing is the family of Transforming growth factors or TGFs. TGF.alpha. is upregulated in some human cancers. It is produced in macrophages, brain cells, and keratinocytes, and induces epithelial development. TGF.beta subtypes (beta-1, beta-2 and beta-3) are upregulated in some human cancers, and play crucial roles in tissue regeneration, cell differentiation, embryonic development, and regulation of the immune system.

TGF.beta. receptors are single pass serine/threonine kinase receptors. These proteins were originally characterized by their capacity to induce oncogenic transformation in a specific cell culture system, namely rat kidney fibroblasts. Application of the transforming growth factors to normal rat kidney fibroblasts induces cultured cells to proliferate and overgrow, no longer subject to the normal inhibition caused by contact between cells.

In one embodiment, the filler composition may include one or more growth factors selected from the group comprising TGF-alpha, TGF-beta 1, TGF-beta-2, TGF beta-3. The composition may also additionally include other growth factors or their subtypes or recombinant versions including but not limited to vascular epithelial growth factor (VEGF), fibroblast growth factor (FGF), never growth factor (NGF), epidermal growth factor (EGF), bone morphogenetic proteins (BMPs), platelet-derived growth factor (PDGF) or any combinations thereof. The concentration of one or more growth factor in the composition may range from about 50 ng/ml to about 1 mg/ml.

Use of the Composition to Prevent Microbial Infections

Often contaminated or impure material used for soft tissue augmentation can result in a clustered outbreak of infection or foreign body reaction which can negatively impact the robustness of wound healing. Accordingly, the composition of the present disclosure include a first component which is an anti-microbial solution that includes an anti-microbial agent in concentrations from about 0.1% to about 10% of the total filler composition. Although any known common antibiotics like gentamicin, norfloxacin, cefazolin, amikacin, vancomycin or compounds such as nitric oxide that are known to have bactericidal properties can be used in the composition, in one embodiment the anti-microbial agent may be Taurolidine. Taurolidine is an anti-microbial agent that has both anti-microbial and anti-lipopolysaccharide properties.

Use of the Composition in Soft Tissue Augmentation

Soft tissue augmentation can be produced with the introduction of active ingredients along with the existing marketed products or autologous tissues isolated either through liposuction or other means. Soft-tissue fillers, which can include, for example, injectable collagen, fat or hyaluronic acid (HA), can help fill in lines and creases that are a consequence of the aging process, temporarily restoring a smoother, more youthful-looking appearance. When injected beneath the skin, fillers plump up creased and sunken areas of the face. They can also add fullness to the lips and cheeks. Injectable fillers may be used alone or in conjunction with a resurfacing procedure, such as a laser treatment, or a re-contouring procedure, such as a facelift.

The filler composition of the present disclosure includes a first component comprising an anti-microbial agent in amounts ranging from about 0.1% to about 10% of the total filler composition and a second component comprising at least one growth factor, stem cells in a combined amount ranging from about 0.01% to about 5% of the total filler composition, and carrier which is crosslinked with a crosslinking agent present in amounts ranging from about 1% to about 5% of the total filler composition. The carrier in the composition may be any known filler agent including but not limited to hylauron. The composition may additionally include any physiologically acceptable solution or buffer in amounts ranging from about 95% to about 80% of the total filler composition.

Hyaluronan (also called hyaluronic acid or hyaluronate) is a non-sulfated glycosaminoglycan distributed widely throughout connective, epithelial, and neural tissues. Hyaluronan is one of the chief components of the extracellular matrix; it contributes significantly to cell proliferation and migration, and may also be involved in the progression of some malignant tumors. The average 70 kilogram (kg) man has roughly 15 grams of hyaluronan in his body, one third of which is turned over (that is, degraded and synthesized) every day.

Hyaluronan is naturally found in many tissues of the body such as, for example, skin, cartilage, and the vitreous humor. It is therefore well suited to biomedical applications targeting these tissues. Hyaluronan biomedical products have been approved for use in, for example, eye surgery (that is, corneal transplantation, cataract surgery, glaucoma surgery and surgery to repair retinal detachment) and ophthalmic surgery.

Hyaluronan is also used to treat osteoarthritis of the knee. Such treatments are administered as a course of injections into the knee joint and are believed to supplement the viscosity of the joint fluid, thereby lubricating the joint, cushioning the joint and producing an analgesic effect. It has also been suggested that hyaluronan has positive biochemical effects on cartilage cells. Recently, oral use of hyaluronan has been suggested although effectiveness needs to be demonstrated. Some preliminary clinical studies exist that suggest that oral administration of hyaluronan has a positive effect on osteoarthritis.

Due to its high biocompatibility and its common presence in the extracellular matrix of tissues, hyaluronan is gaining popularity as a biomaterial scaffold in tissue engineering research. Additionally, in some cancers, hyaluronan levels correlate well with malignancy and poor prognosis. Hyaluronan is thus often used as a tumor marker for prostate and breast cancer. It may also be used to monitor the progression of the disease.

Hyaluronan may also be used postoperatively to induce tissue healing, notably after cataract surgery. Current models of wound healing propose that larger polymers of hyaluronic acid appear in the early stages of healing to physically make room for white blood cells, which in turn mediate the immune response. In other embodiments of the present disclosure fillers may also include for example, collagen, fat, alginates, gelatin, collagen-based fillers and poly-L-lactic acid.

The carrier/filler (for example, hyaluronan) present in the composition of the present disclosure may be combined with the anti-microbial agent, for example Taurolidine. The combination of the anti-microbial agent with the carrier component hyaluronan may be done through the use of aqueous or non-aqueous solvents. Once combined, delivery of the composition may be accomplished, via intracutaneous, subcutaneous, intramuscular, or as a bolus.

In another embodiment the carrier/filler may be combined with at least one growth factor such as TGF-beta so that the growth factor may be delivered in a pharmaceutically active state. In some another embodiment, the carrier/filler may be combined with at least one growth factor and stem cells. In this embodiment the combined amount of stem cells and the growth factor may range from about 0.01% to about 5% of the total composition.

It has been known that the introduction of growth hormone (GH) from polymeric blends such as collagen and hyaluronic acid release physiological concentrations of growth hormone. As such, GH influences cellular growth, specifically stimulation of human osteoblast-like cells (HOBs). The researchers measured this effect by getting a sense of cell proliferation and alkaline phosphatase (ALP), which is a biochemical marker of HOB, thereby concluding that cell differentiation is possible. Carriers for these hormones have improved with time, as products such as, for example, Restylane are cross-linked to prolong their lifetime when injected as a filler. For example, in an existing approach, a NASHA version of hyaluronic acid is produced from a microorganism Streptococcus zooepidemicus to produce highly pure hyaluronic acid that is chemically identical to that that exists in the skin and other tissues.

Existing techniques such as, for example, U.S. Pat. No. 5,827,937, which is hereby incorporated by reference, have been established to cross-link hyaluronic acid (HA). It is possible to introduce GH into cross-linked versions of HA in situ and to utilize the hydrogel as a carrier and stabilizer for the hormone. In existing approaches other than those cross-linking versions of hyaluronic acid, it has been shown that HA/Pluronic composite hydrogels could be formed demonstrating thermally reversible swelling, de-swelling behaviors which could regulate the release of rhGH (recombinant human growth hormone). Sustained release of the hormone has been observed from these composite HA structures. Other cross-linking strategies of hyaluronic acid have also been studied as scaffolds, wherein a cross-linking strategy targeting the alcohol groups via a poly(ethylene glycol) diepoxide cross-linker was investigated for the generation of degradable HA hydrogels. For additional support for cell adhesion in vitro, collagen was incorporated into the HA solution prior to the cross-linking process. Additionally, using biotinylation enables one to fashion molecules that have the ability to attach avidin-type biomolecules.

Both CRS (Cimicifuga racemosa Extract) and TNS (Tissue Nutrient Solution Recovery Complex (TNS) (SkinMedica, Carlsbad, Calif., USA), a product containing a variety of growth factors including VEGF, PDGF-A, G-CSF, HGF, IL-6, IL-8, and TGF-b1) provide significant improvement in the appearance of facial rhytides. The other ingredients in CRS alone, minus the TGF-b1, however, fail to demonstrate a statistically significant improvement in rhytides. It is suggested that if the growth factor portion of the product stimulates neocollagenesis and collagen remodeling, then the incorporation of supplemental L-ascorbic acid, a necessary cofactor for collagen synthesis, could enhance this process.

The injection of cross-linked hyaluronic acid is thought to stimulate collagen synthesis. By injecting a hyaluronic acid, NASHA-type product, Restylane, intracellular and extracellular detection of type 1 procollagen synthesis in the skin treated with the NASHA product has been detected as this is a pattern that is consistent with the production of type 1 collagen, from fibroblasts. Additionally, the stimulation of tissue growth factor and transforming growth factor. beta has been found.

As noted above, numerous filling agents or fillers exist. Examples may include the following items. Autologous collagen (fat) works by promoting an inflammatory response that, in turn, results in the deposition of new collagen at the recipient site. Harvested autologous fat from procedures such as, for example, liposuction, is processed by mixing it with sterile distilled water and then allowing it to freeze, thereby leading to the rupture of adipocytes. The liquefied fraction of intracellular triglycerides is then ready to be injected through a fine-gauge needle (for example, a 30-gauge needle) suitable for intradermal injection. This technique is often used in conjunction with subcutaneous fat transplantation.

The benefit of this technique is its autologous nature, negating the need for hypersensitivity testing. However, it requires harvesting the tissue from a donor site as well as involved preparation and expedient administration after harvesting the tissue. Also, studies vary on the duration of autologous collagen. The range is months to years, depending on the methods used in fat harvesting, processing, and transplanting.

Restylane is an FDA-approved non-animal-stabilized hyaluronic acid derivative used for soft tissue augmentation. Unlike Hylaform gel, it is derived from streptococcal bacterial fermentation and does not require an animal source. At 20 milligrams per milliliter (mg/mL), Restylane has a higher concentration of hyaluronic acid than Hylaform gel. It is used to treat rhytids and scars, and is also used in lip augmentation. Restylane correction was noted to be 82% at 3 months and 33% at 1 year in a study involving 285 wrinkles treated in 113 patients.

Perlane is also a hyaluronic acid derivative at 20 mg/mL, but it is a more robust form of Restylane for use in the deep dermis and at the dermal-fat junction. It is used in Canada and has received FDA approval in 2007.

Being hyaluronic acid derivatives similar to Hylaform Gel, Restylane and Perlane have less risk of clumping and can be inserted more smoothly. Overcorrection is not needed with these products.

Sculptra is poly-L-lactic acid and is FDA-approved for the treatment of HIV facial lipoatrophy. It serves as a volume enhancer and is used for indications similar to those for autologous fat transfer. Results are not immediate. Treatment is performed as a series of 3-5 treatments approximately one month apart. Sculptra is marketed in most countries outside the United States. New-fill is a non-animal-derived polylactic acid touted to be biocompatible, biodegradable, and immunologically inert. It is distributed freeze-dried, can be stored at room temperature, and is reconstituted with sterile water. New-fill is injected either into the superficial dermis for the treatment of rhytids and acne scars or subdermally to treat lipodystrophy of the cheeks and hands, liposuction contour deformities, and lip atrophy.

However, all the above existing products and approaches lack longevity associated with administration of each product.

Because the composition of the present invention include growth factors, an anti-microbial agent and stem cells the composition is capable of promoting the longevity of a filler agent by stimulating appropriate cytokines known in wound repair such as, for example, optimizing the proliferative phase of wound repair when there is granulation tissue formation.

Stem Cells for Soft Tissue Augmentation

The composition of the present disclosure may include at least one stem cell to about 7×10⁶ cells stem cells to further promote the process of wound healing. These stem cells may be autologous or non-autologous in origin.

There are three known accessible sources of autologous adult stem cells in humans: Bone marrow, which requires extraction by harvesting, that is, drilling into bone (typically the femur or iliac crest). Adipose tissue (lipid cells), which requires extraction by liposuction. Blood, which requires extraction through apheresis, wherein blood is drawn from the donor (similar to a blood donation), and passed through a machine that extracts the stem cells and returns other portions of the blood to the donor.

Stem cells can also be taken from umbilical cord blood just after birth. Of all stem cell types, autologous harvesting involves the least risk. By definition, autologous cells are obtained from one's own body, just as one may bank his or her own blood for elective surgical procedures.

Embryonic stem (ES) cells are stem cells derived from the inner cell mass of a blastocyst, an early-stage embryo.^{[9]} Human embryos reach the blastocyst stage 4-5 days post fertilization, at which time they consist of 50-150 cells. ES cells are pluripotent and give rise during development to all derivatives of the three primary germ layers: ectoderm, endoderm and mesoderm. In other words, they can develop into each of the more than 200 cell types of the adult body when given sufficient and necessary stimulation for a specific cell type. They do not contribute to the extra-embryonic membranes or the placenta.

Nearly all research to date has made use of mouse embryonic stem cells (mES) or human embryonic stem cells (hES). Both have the essential stem cell characteristics, yet they require very different environments in order to maintain an undifferentiated state. Mouse ES cells are grown on a layer of gelatin as an extracellular matrix (for support) and require the presence of leukemia inhibitory factor (LIF). Human ES cells are grown on a feeder layer of mouse embryonic fibroblasts (MEFs) and require the presence of basic fibroblast growth factor (bFGF or FGF-2). Without optimal culture conditions or genetic manipulation, embryonic stem cells will rapidly differentiate.

A human embryonic stem cell is also defined by the expression of several transcription factors and cell surface proteins. The transcription factors Oct-4, Nanog, and Sox2 form the core regulatory network that ensures the suppression of genes that lead to differentiation and the maintenance of pluripotency. The cell surface antigens most commonly used to identify hES cells are the glycolipids stage specific embryonic antigen 3 and 4 and the keratan sulfate antigens Tra-1-60 and Tra-1-81. The molecular definition of a stem cell includes many more proteins and continues to be a topic of research.

There are currently no approved treatments using embryonic stem cells. The first human trial was approved by the US Food and Drug Administration in January 2009. However, the human trial was not initiated until Oct. 13, 2010 in Atlanta for spinal injury victims. On Nov. 14, 2011 the company conducting the trial announced that it will discontinue further development of its stem cell programs. ES cells, being pluripotent cells, require specific signals for correct differentiation—if injected directly into another body, ES cells will differentiate into many different types of cells, causing a teratoma. Differentiating ES cells into usable cells while avoiding transplant rejection are just a few of the hurdles that embryonic stem cell researchers still face. Many nations currently have moratoria on either ES cell research or the production of new ES cell lines. Because of their combined abilities of unlimited expansion and pluripotency, embryonic stem cells remain a theoretically potential source for regenerative medicine and tissue replacement after injury or disease.

The primitive stem cells located in the organs of fetuses are referred to as fetal stem cells. There are two types of fetal stem cells:

Fetal proper stem cells come from the tissue of the fetus proper, and are generally obtained after an abortion. These stem cells are not immortal but have a high level of division and are multipotent.

Extraembryonic fetal stem cells come from extraembryonic membranes, and are generally not distinguished from adult stem cells. These stem cells are acquired after birth, they are not immortal but have a high level of cell division, and are pluripotent.

In a preferred embodiment the composition of the present disclosure may include at least one stem cell to up to about 7×10⁶ stem cells. The stem cells suitable for the composition may preferably be autologous stem cells derived from a patient in need of soft tissue augmentation. The use of stem cells may provide more long term benefits as new tissue can be generated within the matrix of the tissue augmentation formulation. In this way newly generated tissue will have a more lasting effect.

Preparation of the Composition

A method for preparing a filler composition for soft tissue augmentation include the steps of: providing a first component comprising an anti-microbial agent; providing a second component comprising at least one growth factor, stem cells, and a carrier; wherein the carrier is cross-linked with a cross-linking agent; and combining the first component and the second component so that the anti-microbial in the first component improves the therapeutic effect by about 50% and prolong the useful life of the composition when injected in a target tissue compared to a composition that does not include the anti-microbial agent.

Also, the anti-microbial agent in the composition may have a concentration from about 0.1% to about 10% of the total composition while the growth factor may be present in a concentration ranging from about 50 nanograms per milliliter (ng/ml) to about one milligram per milliliter (mg/ml). The carrier may include, for example, hyaluronan, and the cross-linking agent may include a sugar. Exemplary sugars can include ribose, glycerose, threose, erythrose, lyxose, xylose, arabinose, ribose, allose, altrose, glucose, mannose, gulose, idose, galactose, talose, a naturally occurring reducing sugar (for example, a diose, a triose, a tetrose, a pentose, a hexose, a septose, an octose, a nanose, and/or a decose), and/or a disaccharide, (for example, maltose, lactose, sucrose, cellobiose, gentiobiose, melibiose, turanose, and/or trehalose).

The techniques for producing a composition for augmenting soft tissue include the steps of obtaining the anti-microbial, preparing a first component which is an aqueous solution containing the anti-microbial in citrate buffer, obtaining a carrier, cross-linking the carrier with a cross-linking agent to form a carrier mix, adding the growth factor and stem cells to the carrier mix (building the carrier mix) and combining the first component and the second component into a combination having a therapeutic effect.

In some embodiments, the anti-microbial agent may be combined with the cross-linked carrier via use of aqueous solvents and/or non-aqueous solvents. Accordingly the composition may additionally include aqueous or non-aqueous solvents, buffers or other excipients or may include natural proteins or synthetic peptides, insulin and/or hormones. natural proteins or synthetic peptides, anti-infective drugs such as steroidal anti-inflammatory drugs or non-steroidal anti-inflammatory drugs (NSAIDS).

Methods for Using the Composition

The method of using the composition of the present disclosure include the steps of: preparing a therapeutic composition, wherein the composition includes a first component comprising an anti-microbial agent; a second component comprising at least one growth factor, stem cells, a carrier, wherein the carrier is cross-linked with a crosslinking agent; and using the cross-linked carrier to deliver the composition to a target tissue; and administering the composition to a target tissue in a subject. The administration of a therapeutically effective amount of a composition to a patient, includes at least one of administering a composition to a patient intracutaneously, subcutaneously, intramuscularly and/or as a bolus.

Additionally, the method for delivering a composition can include the steps of cross-linking a carrier with a cross-linking agent, incorporating an anti-microbial into the cross-linked carrier, and using the cross-linked carrier to deliver the anti-microbial to a target site. The anti-microbial can also be combined with a protein, a peptide and/or a growth factor or autologous or non-autologous stem cells.

Although illustrative embodiments of the present invention have been described herein, it is to be understood that the invention is not limited to those precise embodiments, and that various other changes and modifications may be made by one skilled in the art without departing from the scope or spirit of the invention.

### EXAMPLES

Preparation of an Anti-Microbial Solution

In an exemplary embodiment, Taurolidine solution can be prepared as follows; In a standard 250 ml flask a 100 ml solution 3.5 g sodium citrate is added to make a sodium citrate buffer solution. To this 1.35 g of Taurolidine is added to prepare a 1.35% Taurolidine solution.

HA Matrix Containing TGF-Beta

The hyaluronic acid (HA) can be prepared with TGF-β as follows. In a standard 3 neck 500 ml organic reaction kettle with a safe-lab stirrer bearing, 1 millimole (mmole) of sodium hyaluronate (HA), 1.1 mMoles ribose were added to water to form a 1% carrier mix solution. The HA and ribose were completely dissolved in the water, resulting in the formation of a viscous liquid adjusted to a pH of 6 to 7. To that solution, about 0.1% weight/weight (w/w) (based on solids), TGF-β is added and stirred.

The above solution is incubated at 37° C. to allow for association of the sugar, growth factor and hyaluronic acid anywhere from 2 hours to 200 hours. This results in a composition that can be further processed to a formulation that can be freeze-dried. The resulting mixture may be freeze-dried to yield a mixture of the HA and the growth factor. This can then be reconstituted for injection.

HA Matrix Containing Insulin

In another exemplary embodiment, hyaluronic acid (HA) can be prepared with insulin as follows. In a standard 3 neck 500 ml organic reaction kettle with a safe-lab stirrer bearing. 1 mMole of sodium hyaluronate (HA), 1.1 mMoles ribose were added to water to form a 1% solution. The HA and ribose were completely dissolved in the water, resulting in the formation of a viscous liquid with a pH of 6 to 7. To that solution, 1000 units of insulin is added and stirred.

The above solution is incubated at 37° C. to allow for association of the sugar, growth factor and hyaluronic acid anywhere from 2 hours to 200 hours. This results in a composition that can be further processed to a formulation that can be freeze-dried. The resulting mixture may be freeze-dried to yield a mixture of the HA and the insulin. This can then be reconstituted for injection.

HA Matrix Containing an Anti-Microbial Agent

In another exemplary embodiment, hyaluronic acid (HA) can be prepared with Taurolidine as follows. In a standard 3 neck 500 ml organic reaction kettle with a safe-lab stirrer bearing. 1 mMole of sodium hyaluronate (HA), 1.1 mMoles ribose were added to water to form a 1% solution. The HA and ribose were completely dissolved in the water, resulting in the formation of a viscous liquid with a pH of 6 to 7. To that solution, 1.35 g Taurolidine solution is added and stirred.

The above solution is incubated at 37° C. to allow for association of the sugar, Taurolidine and hyaluronic acid anywhere from 2 hours to 200 hours. This results in a composition that can be further processed to a formulation that can be freeze-dried. The resulting mixture may be freeze-dried to yield a mixture of the HA and the insulin. This can then be reconstituted for injection.

Additionally, yet another exemplary embodiment can use all of the elements of the two examples above, except that in lieu of incubation, the anti-microbial solution is mixed with HA-ribose and the HA-ribose may be exposed to low levels of eBeam radiation or ultraviolet light to promote cross-linking of the composition with the anti-microbial agent. In this situation, less than 1 megaelectron volt (MeV) may be utilized at very short duration of 10 seconds or less.

Alternatively, an ultraviolet irradiating device (3 kilowatt (kW) metal halide lamp) may be used for less than five minutes of exposure insuring the solution never exceeds 37° C. This can be repeated until the desired level of cross-linking is achieved.

Further, the techniques described here can also include the use of resveratrol (a phytoalexin found, for example, in grapes and other plants). By way of example, the amount of resveratrol in food can vary, as wine, for example, contains between 0.2 and 40 milligrams per liter (mg/L) of resveratrol.

HA Lattice Containing Stem Cells

One or more embodiments of the present invention also include incorporating adipose-derived stem cells in a HA lattice and/or matrix. Autologous fat can be harvested from a patient and adipose-derived stem cells can be extracted therefrom. By way of example, about 250 cubic centimeters (CCs) of autologous fat can be harvested from a patient, and up to 75×10⁶ adipose-derived stem cells can be extracted from the autologous fat. Additionally, up to 75×10⁶ adipose-derived stem cells can be incorporated into, for example, 1 to 10 CCs of hyaluronic acid (HA) lattice.

At least one embodiment of the invention may provide one or more beneficial effects, such as, for example, stimulating appropriate cytokines known in wound repair such as, for example, optimizing the proliferative phase of wound repair when there is granulation tissue formation.

Evaluations of the Performance of the Anti-Microbial Hyaluronic Gels

By way of Examples, the following evaluations were done to assess the performance of the antimicrobial hyaluronic acid gels. Solution exposure of the antimicrobial hyaluronic acid hydrogels immersed in early phase concentrations of 2 test microorganisms were conducted. Additionally, the physical properties of the hydrogels were assessed to determine their shear/thixotropic properties.

Anti-Bacterial, Hyaluronic Acid Hydrogel Preparation

Formulations of taurolidine in aqueous solutions of HA crosslinked with 1,4-butanediol diglycidyl ether (BDDE) were prepared for evaluating its impact on the microbial killing effects. Three concentrations 1.5%, 3% and 6% of taurolidine were formulated in aqueous solutions of crosslinked HA of three molecular weights: low molecular weight (LMW) 21-40 kDa, medium molecular weight (MMW) 310-450 kDa and high molecular weight (HMW) 750 kDa-1.0 MDa. Control formulations were prepared without addition of the drug. The compositions of each formulation are given in Table 1 below.

TABLE 1

| | | | | | | |
|---|---|---|---|---|---|---|
| | | | Drug | BDDE | BDDE/HA | |
| Sample Name | HA | HA (%) | (%) | (%) | (%) | pH |

Controls (no drug)

| | | | | | | |
|---|---|---|---|---|---|---|
| 13079-1 | LMW | 2.0 | 0 | 1.0 | 0.5 | 7.2 |
| 13079-2 | MMW | 2.0 | 0 | 0.9 | 0.5 | 7.0 |
| 13079-3 | HMW | 2.0 | 0 | 0.9 | 0.5 | 7.5 |

Formulation with 1.5% Taurolidine

| | | | | | | |
|---|---|---|---|---|---|---|
| 13079-4 | LMW | 2.0 | 1.5 | 0.9 | 0.5 | 8.0 |
| 13079-5 | MMW | 1.9 | 1.5 | 0.9 | 0.5 | 7.6 |
| 13079-6 | HMW | 1.9 | 1.5 | 0.9 | 0.5 | 7.9 |

Formulation with 3.0% Taurolidine

| | | | | | | |
|---|---|---|---|---|---|---|
| 13079-7 | LMW | 2.0 | 2.9 | 0.9 | 0.4 | 7.0 |
| 13079-8 | MMW | 2.0 | 3.0 | 0.9 | 0.5 | 7.3 |
| 13079-9 | HMW | 2.0 | 2.9 | 0.9 | 0.5 | 7.7 |

Formulation with 6% Taurolidine

| | | | | | | |
|---|---|---|---|---|---|---|
| 13079-10 | LMW | 2.0 | 6.1 | 1.0 | 0.5 | 7.3 |
| 13079-11 | MMW | 2.0 | 6.0 | 0.9 | 0.5 | 7.8 |
| 13079-12 | HMW | 2.0 | 6.1 | 1.0 | 0.5 | 7.7 |

Solution Exposure of Hyaluronic Acid to Living Cultures

### Example 1

In this study, two strains of bacteria, namely Pseudomonas aeruginosa (PAO1) and Stapylococcus aureus (SA 113) were evaluated for total kill of each of the strains after 4 and 24 hours of exposure to the taurolidine-HA formulation. 20 μl of early phase culture of each strain was placed in 980 μl of each Hyaluronic Acid formulation. Samples of remaining bacteria concentrations were determined for the samples after 4 and 24 hours of exposure. Two controls (with and without HA), and HA gels with 3 different drug concentrations were evaluated. They were also evaluated against a known antimicrobial, gentamicin.

TABLE 2

| | | | | | | | | |
|---|---|---|---|---|---|---|---|---|
| | PA01 | PA01 | SA113 | SA113 | | | | |
| Sample | (4 hr) | (24 hr) | (4 hr) | (24 hr) | STDV1 | STDV1 | STDV1 | STDV1 |

| | | | | |
|---|---|---|---|---|
| Control | 1.40E+08 | 1.40E+08 | 1.72E+08 | 1.72E+08 |
| (No HA and No | | | | |
| Drug) | | | | |

With HA but No Drug

| | | | | | | | | |
|---|---|---|---|---|---|---|---|---|
| 13079-1 | 7.63E+03 | 0.00E+00 | 0.00E+00 | 0.00E+00 | 1.07E+04 | 0.00E+00 | 0.00E+00 | 0.00E+00 |
| (LMW HA) | | | | | | | | |
| 13079-2 | 2.02E+06 | 0.00E+00 | 1.44E+05 | 0.00E+00 | 9.99E+05 | 1.67E+04 | 0.00E+00 | 0.00E+00 |
| (MMW HA) | | | | | | | | |
| 13079-3 | 2.84E+06 | 2.51E+05 | 2.24E+05 | 0.00E+00 | 7.31E+04 | 1.78E+05 | 2.87E+05 | 0.00E+00 |
| (HMW HA) | | | | | | | | |

With HA and With Drug

| | | | | | | | | |
|---|---|---|---|---|---|---|---|---|
| 13079-4 | 0.00E+00 | 0.00E+00 | 0.00E+00 | 0.00E+00 | 0.00E+00 | 0.00E+00 | 0.00E+00 | 0.00E+00 |
| (LMW HA) | | | | | | | | |
| 13079-5 | 0.00E+00 | 0.00E+00 | 0.00E+00 | 0.00E+00 | 0.00E+00 | 0.00E+00 | 0.00E+00 | 0.00E+00 |
| (MMW HA) | | | | | | | | |
| 13079-6 | 0.00E+00 | 0.00E+00 | 0.00E+00 | 0.00E+00 | 0.00E+00 | 0.00E+00 | 0.00E+00 | 0.00E+00 |
| (HMW HA) | | | | | | | | |
| 13079-7 | 0.00E+00 | 0.00E+00 | 0.00E+00 | 0.00E+00 | 0.00E+00 | 0.00E+00 | 0.00E+00 | 0.00E+00 |
| (LMW HA) | | | | | | | | |
| 13079-8 | 0.00E+00 | 0.00E+00 | 0.00E+00 | 0.00E+00 | 0.00E+00 | 0.00E+00 | 0.00E+00 | 0.00E+00 |
| (MMW) | | | | | | | | |
| 13079-9 | 0.00E+00 | 0.00E+00 | 0.00E+00 | 0.00E+00 | 0.00E+00 | 0.00E+00 | 0.00E+00 | 0.00E+00 |
| (HMW HA) | | | | | | | | |
| 13079-10 | 0.00E+00 | 0.00E+00 | 0.00E+00 | 0.00E+00 | 0.00E+00 | 0.00E+00 | 0.00E+00 | 0.00E+00 |
| (LMW HA) | | | | | | | | |
| 13079-11 | 0.00E+00 | 0.00E+00 | 0.00E+00 | 0.00E+00 | 0.00E+00 | 0.00E+00 | 0.00E+00 | 0.00E+00 |
| (MMW HA) | | | | | | | | |
| 13079-12 | 0.00E+00 | 0.00E+00 | 0.00E+00 | 0.00E+00 | 0.00E+00 | 0.00E+00 | 0.00E+00 | 0.00E+00 |
| (HMW HA) | | | | | | | | |
| Gentamicin | 0.00E+00 | 0.00E+00 | 3.77E+02 | 8.33E+00 | 0.00E+00 | 3.77E+01 | 0.00E+00 | 2.36E+00 |

The results in Table 2 shows that all samples of hydrogels containing the drug, taurolidine provided total kill of both strains of bacteria, Pseudomonas aeruginosa (PAO1) and Stapylococcus aureus (SA 113) after only 4 hours of exposure as compared to the controls (shown in the table by shaded cells).

The thixotropic properties or the viscosity of the formulations at a frequency of 1 Hz was measured using standard viscometers and the results are shown in Table 3 given below.

TABLE 3

| | |
|---|---|
| Formulation | Viscosity in Pa · s |

Controls (no drug)

| | |
|---|---|
| 13079-1 (LMW HA) | 0.01 |
| 13079-2 (MMW HA) | 0.09 |
| 13079-3 (HMW HA) | 0.21 |

Formulation with 1.5% Taurolidine

| | |
|---|---|
| 13079-4 (LMW HA) | 0.01 |
| 13079-5 (MMW HA) | 0.13 |
| 13079-6 (HMW HA) | 0.40 |

Formulation with 3% Taurolidine

| | |
|---|---|
| 13079-7 (LMW HA) | 0.01 |
| 13079-8 (MMW HA) | 0.25 |
| 13079-9 (HMW HA) | 1.47 |

Formulation with 6% Taurolidine

| | |
|---|---|
| 13079-10 (LMW HA) | 0.01 |
| 13079-11 (MMW HA) | 0.55 |
| 13079-12 (HMW HA) | 6.68 |

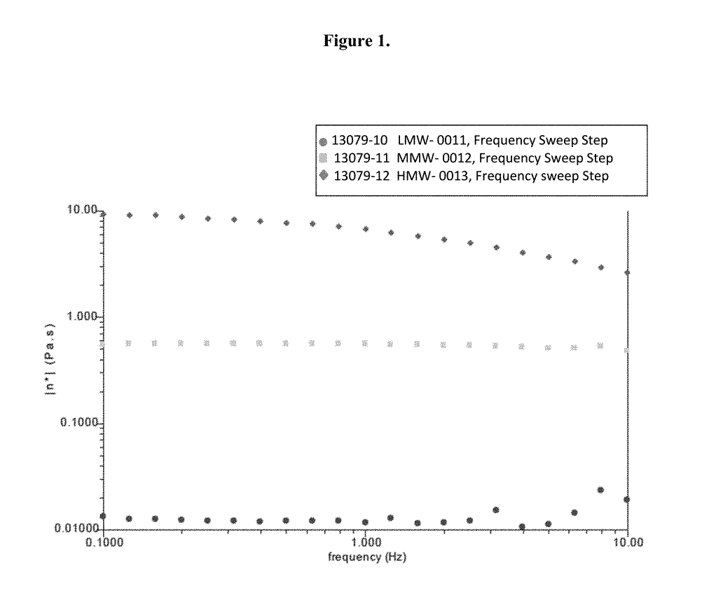

As indicated in Table 3 above, high MW versions (HMW) of Hyaluronic Acid (HA) containing drug had enhanced thixotropic properties by measure of complex viscosity giving values of 0.21, 0.4 and 1.47, and 6.68 Pa seconds at drug concentrations of 0, 1.5, 3, and 6% respectively which increased with drug concentration. This is also illustrated in FIG. 1.

Medium MW (MMW) versions of Hyaluronic Acid also exhibited thixotropic properties to a lesser degree with increasing drug concentration with values of 0.09, 0.13, 0.25, 0.55 Pa seconds at drug concentrations of 0, 1.5, 3 and 6%, respectfully.

All the low MW (LMW) Hyaluronic Acid (HA) gels that exhibited values of 0.01 Pa seconds for all the samples and did not exhibit thixotropic properties.

Although illustrative embodiments of the present invention have been described herein, it is to be understood that the invention is not limited to those precise embodiments, and that various other changes and modifications may be made by one skilled in the art without departing from the scope or spirit of the invention.

1. A therapeutic filler composition capable of being injected into a patient's soft tissue, the composition comprising:
an anti-microbial agent present in amounts ranging from about 0.1% to about 10% of the total composition;
at least one growth factor,
stem cells, and
a carrier;
wherein the therapeutic filler composition is configured to prolong the useful life of the therapeutic filler composition in a target tissue compared to a filler composition that does not include an anti-microbial agent.

2. The filler composition of claim 1, further comprising one or more hormones, natural proteins or synthetic peptides, anti-infective drugs, aqueous or non-aqueous solvents, buffers or other excipients.

3. The filler composition of claim 1, wherein the anti-microbial agent is taurolidine.

4. The filler composition of claim 1, wherein the concentration of the at least one growth factor and stem cells range from about 0.01% to about 5% of the total composition.

5. The filler composition of claim 1, wherein the growth factor may be selected from the group comprising vascular epidermal growth factor, Fibroblast growth factors, bone morphogentic proteins, transforming growth factor-beta, platelet-derived growth factor, nerve growth factor or any combinations thereof.

6. The filler composition of claim 5, wherein the growth factor is transforming growth factor-beta.

7. The filler composition of claim 1, wherein the stem cells may be autologous or non-autologous.

8. The filler composition of claim 1 wherein the carrier is crosslinked with a sugar.

9. The filler composition of claim 1, wherein the carrier is present in amounts ranging from about 1% to about 5% of the total composition.

10. The filler composition of claim 9, wherein the carrier is derived from fat, dermis, or collagen.

11. (canceled)

12. The filler composition of claim 8 wherein the carrier is selected from the group consisting of hyaluronic acid, silicone oil, expanded polytetrafluoroethylene, polymethacrylate, polylactones, calcium hydroxyapatite, alkyl-imide gel polymer, degradable polymers, or combinations thereof.

13. The filler composition of claim 12, wherein the carrier comprises a degradable polymer comprising glycolide, lactide, e-caprolactone, p-dioxinone, and trimethylene carbonate.

14. (canceled)

15. (canceled)

16. (canceled)

17. (canceled)

18. A method for preparing a filler composition for soft tissue augmentation comprising the steps of:
providing a first component comprising an anti-microbial agent;
providing a second component comprising at least one growth factor, stem cells, and a carrier; wherein the carrier is cross-linked with a cross-linking agent; and
combining the first component and the second component in an amount sufficient to improve the therapeutic effect of the filler composition and prolong the life of the filler composition when injected in a target tissue compared to a filler composition that does not include the anti-microbial agent.

19. The method of claim 18, wherein the anti-microbial agent is Taurolidine.

20. The method of claim 18, wherein the at least one growth factor is transforming growth factor-beta and is present in concentration ranging from about 50 ng/ml to 1 mg/ml.

21. The method of claim 18, wherein the stem cells are autologous cells.

22. The method of claim 18, wherein the carrier is a hyaluronic acid.

23. The method of claim 18, wherein the cross-linking agent is a sugar.

24. (canceled)

25. (canceled)

26. (canceled)

27. (canceled)

28. (canceled)

29. (canceled)

30. The method of claim 18, wherein the therapeutic filler composition is administered through intracutaneous, subcutaneous or intramuscular routes.
